(19) United States
(12) Patent Application Publication
(10) Pub. No.: US 2022/0143359 A1
(43) Pub. Date: May 12, 2022

(54) ANTI-DROWSINESS DEVICE

(71) Applicants: Riko YOHENA, Saitama Pref. (JP); Kin Fung Wong, Tokyo (JP); Masako TOBARI, Saitama Pref (JP); Yudai KAWAWADA, Saitama Pref. (JP); Nan WU, Tokyo (JP); Yu LI, Tokyo (JP)

(72) Inventors: Riko YOHENA, Saitama Pref. (JP); Kin Fung Wong, Tokyo (JP); Masako TOBARI, Saitama Pref (JP); Yudai KAWAWADA, Saitama Pref. (JP); Nan WU, Tokyo (JP); Yu LI, Tokyo (JP)

(21) Appl. No.: 17/553,820

(22) Filed: Dec. 17, 2021

(51) Int. Cl. A61M 21/00; A61N 1/04; A61N 1/36; A61N 1/08; A61H 9/00; B60W 40/08; B60W 50/14

(52) U.S. Cl. CPC A61M 21/00; B60W 2540/229; A61N 1/36025; A61N 1/08; A61H 9/0071; A61N 1/36003; A61N 1/36031; B60W 40/08; B60W 50/14; A61M 2021/0077; A61M 2230/10; A61M 2021/0022; A61H 2205/106; A61M 2205/18; A61M 2021/0083; A61M 2021/0055; A61H 2201/105; A61N 1/0484

(57) ABSTRACT

An anti-drowsiness device includes a head cover; an electroencephalograph, which is formed on the head cover, the electroencephalograph receiving the brain-waves all the time and generating a sleep-detection signal when a specific frequency, which shows drowsiness, is received, and an electrostimulator, which is formed on the head cover, the electrostimulator generating electrostimulation when receiving the sleep-detection signal.

### BACKGROUND OF THE INVENTION

### 1. Field of the Invention

This invention has been made by the members of MIMURA Seminar, Graduate School of Law, Nihon Univ. as a team. The present invention relates to an anti-drowsiness device, particularly an anti-drowsiness device that removes a person's drowsiness by applying any physical, electrical, or chemical stimulations to the human body in response to human movement or brain waves.

### 2. Description of the Related Arts

Conventionally, there is an anti-drowsiness device that removes a person's drowsiness by applying any physical or electrical stimulations in response to the movement of the person. The conventional anti-drowsiness device has been used while driving a car. For example, such a conventional anti-drowsiness device has a switch and an electrode, the switch is connected to a seat belt or the like, and the electrode is attached to an earlobe. The conventional anti-drowsiness device is designed to prevent drowsiness by sending a weak electric current to the earlobes when the driver presses a switch on the seat belt.

However, the conventional anti-drowsiness device has a problem that it is difficult for the driver to press the switch when he or she feels drowsy because the driver manually presses the switch by himself/herself.

According to the present invention, the above-mentioned problem can be solved. And the device can automatically apply any physical or electrical stimulations to the human body instead of manually before the person suffers from drowsiness, especially by sensing brain-waves.

Further, some improved conventional devices give some simulations automatically. But, the improved conventional devices are activated by sensing the human movement or the head tilt. However, it is difficult to adjust the sensing condition for the tilt of the head, and even if the user has no drowsiness, the electric stimulation is applied to the human only by tilting the head a little.

### SUMMARY OF THE INVENTION

According to the present invention, the above-mentioned problem can be solved, and detecting brain-waves with higher accuracy in detecting drowsiness than sensing in tilting the head, it is possible to give electrical stimulation when the person is really drowsy.

### BRIEF DESCRIPTION OF THE DRAWINGS

The invention will be described with reference to the accompanying drawing, in which:

FIG. 1 shows an overall side view of an anti-drowsiness device, according to the first embodiment of the present invention:

FIG. 2 shows an overall schematic diagram of the anti-drowsiness device, according to the second embodiment of the present invention;

FIG. 3 shows a perspective view of a physical stimulant spray device, according to the second embodiment of the present invention;

FIG. 4 shows a perspective view of a physical stimulation massage machine, according to the second embodiment of the present invention;

FIG. 5 shows an overall schematic diagram of the anti-drowsiness device, according to the third embodiment of the present invention.

### DETAILED DESCRIPTION OF THE PREFERRED EMBODIMENTS

### First Preferred Embodiment

Preferred embodiments of the present invention are described below with reference to the drawings. In each drawing, the same reference numerals indicate the same or similar components passing through all the embodiments.

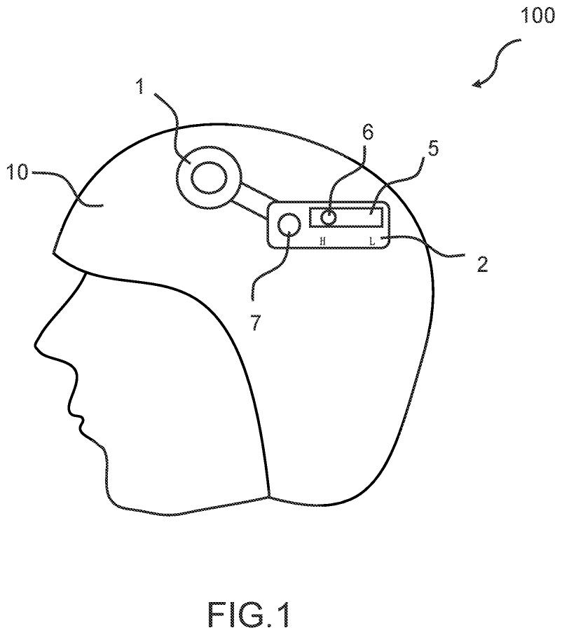

FIG. 1 is an overall schematic diagram of an Anti-Drowsiness device 100 according to the first embodiment of the present invention. This anti-drowsiness device 100 is provided with a head cover 10, an electroencephalograph 1 formed on the head cover 10, and an electrostimulator 2. The electroencephalogram 1 is electrically connected to the electrostimulator 2. The electroencephalograph 1 always receives brain-waves emitted from the frontal lobe, for example, and transmits a sleep-detection signal to the electrostimulator 2 when the brain-waves show 7 to 8 Hz. The brain-waves of the person is at a level of 7 to 8 Hz to show the drowsiness.

The electrostimulator 2 has a function of generating a 0.5 mA˜3 mA current on the scalp when receiving the sleep-detection signal transmitted from the electroencephalograph 1, and generates an electric signal to the scalp. The electroencephalograph 1 is arranged, for example, in the vicinity of the head top part, and the electrostimulator 2 is arranged in the vicinity of the electroencephalograph 1. The electrostimulator 2 also has a meter 5 capable of changing signal output strength and a light 7. The meter 5 includes a slidable stimulation intensity setting member 6, and when a strong stimulation is desired, the stimulation intensity setting member 6 is slided to a “high (H)”position.

By a well-known method, when the electroencephalograph 1 senses the brain-waves of 7-8 Hz, the electrostimulator 2 sends a sleep-detection signal. BY applying this signal, a light 7 formed on the electrostimulator 2 emits green light. The electrostimulator 2 receiving the signal gives electrostimulation to the scalp. Thus, sleepiness is gone. Also, by the light emission of the light 7, a person near the user recognizes that the anti-drowsiness device 100 is activated, and it can be understood that the user is sleeping. When the light is on for a certain amount of time, the color of the light changes from green to red.

For example, when it exceeds one minute, the color is changed to red.

### Second Preferred Embodiment

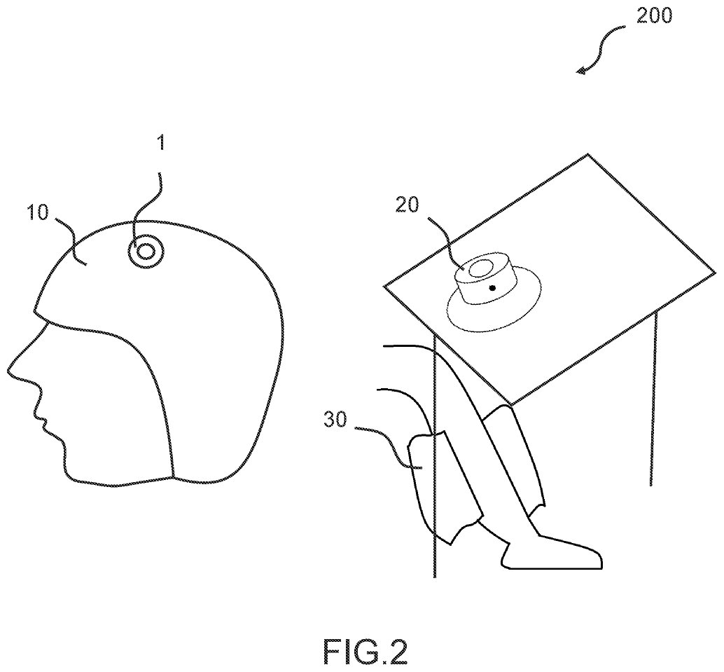

FIG. 2 shows an overall schematic diagram of the anti-drowsiness device, according to the second embodiment of the present invention.

The anti-drowsiness device 200 includes a head cover 10, an electroencephalograph 1 formed on a head cover 10, a physical stimulant spray device 20 for spraying stimulant liquid or stimulant gas, and a physical stimulation massage machine 30. The electroencephalograph 1 is connected to the physical stimulant spray device 20 and the physical stimulation massage machine 30 via Bluetooth. The electroencephalograph 1 always receives brain-waves emitted from the frontal lobe, for example, and transmits a sleep-detection signal to the physical stimulant spray device 20 and the physical stimulation massage machine 30 when the brain-waves show 7 to 8 Hz.

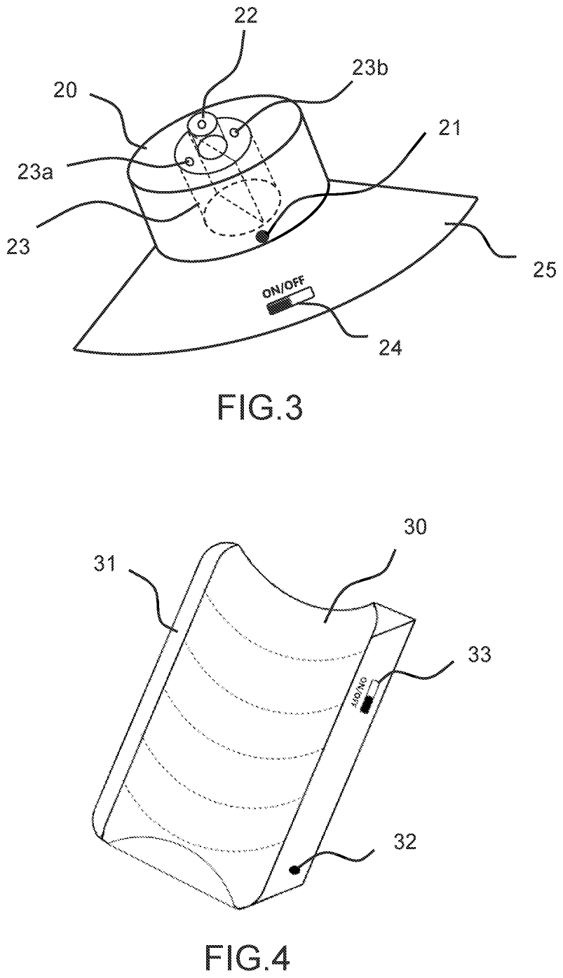

FIG. 3 shows a perspective view of a physical stimulant spray device 20 used in the anti-drowsiness device 200, according to the second embodiment of the present invention. The physical stimulant spray device 20 includes a sleep-detection signal receiver 21, a nozzle 22, a liquid-gas storage container 23, a liquid filling port 23a, a gas filling port 23b, a switch 24 and a suction cup 25. The physical stimulant spray device 20 can be placed anywhere. The suction cup 25 can be installed at any angle, and it's removable. The liquid-gas storage container 23 could be separated from the physical stimulant spray device 20 and could be filled through the liquid filling port 23a and the gas filling port 23b when the liquid or gas is closed to empty. When a sleep-detection signal receiver 21 receives a sleep-detection signal transmitted from the electroencephalograph 1, the sleep-detection signal is transmitted to the nozzle 22, and the nozzle 22 sprays the liquid or the gas containing mint on the human face to awaken the user.

FIG. 4 shows a perspective view of the physical stimulation massage machine 30 used in the anti-drowsiness device 200, according to the second embodiment of the present invention. The physical stimulation massage machine 30 includes a main body 31, a sleep-detection signal receiver 32 and a switch 33. The sleep-detection signal receiver 32 can be used with the same sleep-detection signal receiver 21 used in the physical stimulant spray device 20 shown in FIG. 3. Also, by setting the frequency of the sleep-detection signal to be the same for the sleep-detection signal receiver 21 and the sleep-detection signal receiver 32, the both of the receivers 21, 32 can simultaneously receive the sleep-detection signal transmitted from the electroencephalograph 1. While the switch 33 could be turned off in the case that the physical stimulus spray device 20 shown in FIG. 3 is enough to awaken the user, it could be turned on in the case that the user expects a feeling that he will have a strong sleep attack, in advance.

The main body 31 has a massage function utilizing the conventional technology using air compression. In order to massage the user's calf, the main body 31 has an airbag to control air filling and air discharging into the airbag in use. In case that the switch 33 is turned on, the sleep-detection signal receiver 32 activates a function of automatically operating the massage machine 30, and then it massages the user's calf when receiving the sleep-detection signal transmitted from the electroencephalograph 1.

### Third Preferred Embodiment

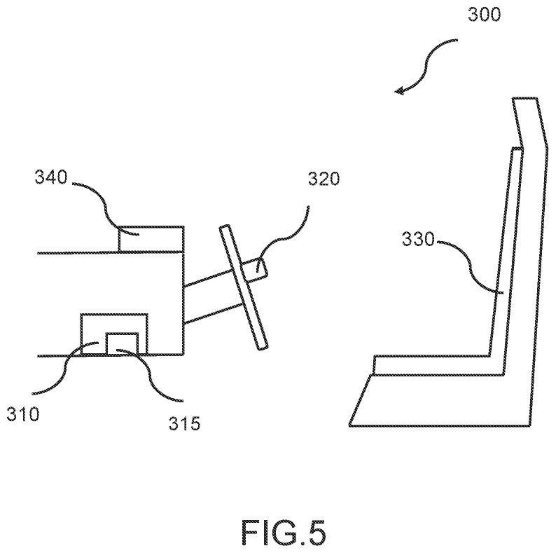

FIG. 5 shows an overall schematic diagram of the anti-drowsiness device, according to the third embodiment of the present invention.

An anti-drowsiness device 300 of the third embodiment is for detecting unusual driving conditions and notifying it to the driver, in particular drowsiness or dozing of the driver, which may make it difficult to continue driving safely.

The anti-drowsiness device 300 includes an event data recorder (EDR) 310, a spray 320 for splaying an high-concentration oxygen 320, an electrostimulator 330 and an alarm generator340. The EDR 310 is equipped with a memory and an AI 315, and records all driving motions as a driving data being stored in the memory. The AI 315 analyzes the data and learns the driver's usual driving habits. By recording this driving behavior, the EDR 310 recognizes what kind of driving habits the driver has when driving safely. When the driver dozes off, an unusual driving operation, which is different from the safe driving operation, is recorded. Therefore, the EDR 310 detects the driver's driving condition by comparing the usual driving behavior recorded in the memory with the unusual driving behavior. The spray 320, the electrostimulator 330, and alarm generator 340 are electrically connected to the EDR 310.

According to the anti-drowsiness device 300 when the EDR 310 detects the unusual driving operation record, the EDR 310 initially generates the sleep-detection signal, and outputs it to the alarm generator 340. The alarm generator 340 generates an alarm, and alerts the driver when it receives the sleep-detection signal. In case that the driver notices the alarm and goes back to the normal driving condition. The EDR 310 recognizes such a condition, and then it will stop transmitting the sleep-detection signal. As a result, the operation of the alarm generator 340 is also stopped, and the alarm is stopped.

On the other hand, when the driver ignores the alarm and the unusual driving operation is maintained, the sleep-detection signal will be outputted continuously. Therefore, when the sleep-detection signal has been outputted for a certain period of time, the sleep-detection signal is then outputted to the spray 320. When the spray 320 receives the sleep-detection signal, it will spray the high-concentration oxygen to the driver's face. The spray 320 can surely reach the driver by installing it on the center of a steering wheel. The oxygen awakes the driver, and even more refreshes the driver. Also, the oxygen will be penetrated into the body and it will be expected to have the effect of activating human's cells and leading the body to vitality.

Furthermore, in order to ensure the effect, when the sleep-detection signal has been outputted to the spray 320, it is also outputted to the electrostimulator 330, which is placed to the driver's seat, so that it is possible to give the drivers back and the driver's bottom muscles electrical stimulation. Thus, it makes the driver's muscles active, stimulates the flow of blood, and accelerates blood circulation. As a result, sleepiness is gone.

In the third preferred embodiment, instead of using the brain-waves, which shows drowsiness, by detecting the driver's unusual driving condition, the anti-drowsiness device 300 encourages the driver to drive safely. On the other hand, in the first embodiment and the second preferred embodiment, since the brain-waves are used, it is possible to use in any occasion other than driving.

While the invention has been described with reference to illustrative embodiments, this description is not intended to be construed in a limiting sense. Thus, shapes, size and physical relationship of each component are roughly illustrated so the scope of the invention should not be construed to be limited to them. Further, to clarify the components of the invention, hatching is partially omitted in the cross-sectional views.

Various other modifications of the illustrated embodiment will be apparent to those skilled in the art in reference to this description. Therefore, the appended claims are intended to cover any such modifications or embodiments as fall within the true scope of the invention.

What is claimed is:

1. An anti-drowsiness device, comprising:
a head cover;
an electroencephalograph, which is formed on the head cover, the electroencephalograph receiving the brain-waves all the time and generating a sleep-detection signal when a specific frequency, which shows drowsiness, is received; and
an electrostimulator, which is formed on the head cover, the electrostimulator generating electrostimulation when receiving the sleep-detection signal.

2. An anti-drowsiness device, as claimed in claim 1, wherein the electroencephalograph outputs the sleep-detection signal to the electrostimulator when it received the brain signal having 7 to 8 Hz.

3. An anti-drowsiness device, as claimed in claim 1, further comprising:
a light, which is formed on the electrostimulator, the light having a function to blink throughout the electrostimulation being applied; and
a stimulation intensity setting member, which is formed on the electrostimulator, the member adjusting the strength of the electrostimulation.

4. An anti-drowsiness device, as claimed in claim 1, wherein the electrostimulator and the electroencephalograph are electrically connected, and are arranged in the vicinity to each other.

5. An anti-drowsiness device, as claimed in claim 2, further comprising:
a light, which is formed on the electrostimulator, the light having a function to blink throughout the electrostimulation being applied; and
a stimulation intensity setting member, which is formed on the electrostimulator, the member adjusting the strength of the electrostimulation.

6. An anti-drowsiness device, as claimed in claim 2, wherein the electrostimulator and the electroencephalograph are electrically connected, and are arranged in the vicinity to each other.

7. An anti-drowsiness device, as claimed in claim 5, wherein the electrostimulator and the electroencephalograph are electrically connected, and are arranged in the vicinity to each other.

8. An anti-drowsiness device, as claimed in claim 3, wherein the electrostimulator and the electroencephalograph are electrically connected, and are arranged in the vicinity to each other.

9. An anti-drowsiness device, as claimed in claim 3, wherein the electrostimulator generates an electrical current having 0.5 to 3 mA in response to the setting of the stimulation intensity setting member.

10. An anti-drowsiness device, as claimed in claim 3, wherein the light changes its color, according to the period of time.

11. An anti-drowsiness device, comprising:
a head cover;
an electroencephalograph, which is formed on the head cover, the electroencephalograph receiving the brain-waves all the time and generating a sleep-detection signal when a specific frequency, which shows drowsiness, is received;
a physical stimulant spray device having a first sleep-detection signal receiver, which receives the sleep-detection signal from the electroencephalograph, the physical stimulant spray device spraying stimulants when the first sleep-detection signal receiver receiving the sleep-detection signal from the electroencephalograph; and
a physical stimulation massage machine equipped with a massage function having a second sleep-detection signal receiver, which is receiving the sleep-detection signal from the electroencephalograph, the physical stimulation massage machine activating the massage function when the second sleep-detection signal receiver receiving the sleep-detection signal from the electroencephalograph.

12. An anti-drowsiness device, as claimed in claim 11, wherein the physical stimulant spray device further comprises,
a liquid-gas storage container; a nozzle for spraying liquid or gas which is formed on the liquid-gas storage container;
a switch for activating the physical stimulant device; and a suction cup, whereby the liquid or gas from the container is sprayed from the nozzle when the first sleep-detection signal receiver receives the sleep-detection signal.

13. An anti-drowsiness device, as claimed in claim 12, wherein the liquid-gas storage container includes a liquid filling port and a gas filling port, whereby the container can be filled from the outside when the liquid or gas is close to empty.

14. An anti-drowsiness device, as claimed in claim 12, wherein the switch can be manually switched between ON and OFF, whereby it is possible to switch to a first mode that the operation of the physical stimulation massage machine is sufficient, or to a second mode that the operation of the physical stimulant spray device is necessary.

15. An anti-drowsiness device, as claimed in claim 12, wherein the physical stimulation massage machine further comprises,
a main body having a massage function, which is able to be installed on an user's calf; a sleep-detection signal receiver; and
a switch for activating the physical stimulation massage machine, whereby the physical stimulation massage machine activates the massage function when the second sleep-detection signal receiver receives the sleep-detection signal from the electroencephalograph.

16. The anti-drowsiness device, as claimed in claim 15, wherein the switch can be manually switched between ON and OFF, whereby it is possible to switch to a first mode that the operation of the physical stimulation massage machine is sufficient, or to a second mode that the operation of the physical stimulant spray device is necessary.

17. An anti-drowsiness device used for a vehicle equipped with a steering wheel and a driver's seat, comprising:
an event data recorder (EDR), which is formed on a vehicle, storing all driving record, the EDR recognizing an usual driving condition by analyzing the driving record, outputting a sleep-detection signal when the EDR detects the unusual driving condition, and stopping the output of the sleep-detection signal when the EDR recognizes the usual driving condition;
an alarm generator, which is electrically connected to the EDR, generating an alarm when receiving the sleep-detection signal from the EDR, stopping the alarm when the sleep-detection signal from the EDR is interrupted,
a spray, which is electrically connected to the EDR, the spray spraying the high-concentration when receiving the sleep-detection signal for a certain period of time after the alarm is generated; and
an electrostimulator, which is connected to the EDR, the electrostimulator generates electrostimulation when receiving the sleep-detection signal for a certain period of time after the alarm is generated.

18. An anti-drowsiness device, as claimed in claim 17, wherein the EDR has a memory, which stores all driving operation as a driving data, and an AI, which analyzes the driving data and instructs the EDR to output the sleep-detection signal when the driving operation shows the unusual condition.

19. An anti-drowsiness device, as claimed in claim 17, wherein the spray, which is installed on the center of the steering wheel.

20. An anti-drowsiness device, as claimed in claim 17, wherein the electrostimulator is placed in a back area and/or a bottom area of the seat.
